(19) United States
(12) Patent Application Publication
(10) Pub. No.: US 2005/0154420 A1
(43) Pub. Date: Jul. 14, 2005

(54) System for terminating heart arrhythmia using electrical shocks delivered through a set of internal and external electrodes configurable to a multitude of shock configurations by selecting the shock vector(s) on a control device that also provides "over shock" safety for patients.

(76) Inventors: Cesar M. Diaz, Rancho Santa Margarita, CA (US); Peter Accorti, Jacksonville, FL (US)

Correspondence Address: Cesar M. Diaz, 17915 Skypark Circle - Ste. D, Irvine, CA, 92614, US

(21) Appl. No.: 10/757,948

(22) Filed: Jan. 14, 2004

(51) Int. Cl. A61N 1/39

(52) U.S. Cl. 607/5

(57) ABSTRACT

The present invention is a system for terminating cardiac arrhythmia using existing defibrillators found in the field in conjunction with a safe junction box. The system is designed to allow the defibrillator to connect to specially designed catheters equipped with specially designed electrodes and external electrodes for coupling energy to the heart that is greatly less than that used with external defibrillation alone. The system has the ability to create internal cardioversion vectors and also “hybrid” cardioversion vectors by allowing the external and internal electrodes to act together in the cardioversion process that is used to terminate arrhythmia in temporary and quasi-permanent implant applications. The quasi-permanent implant applications are greater than thirty day but less than twelve month applications where an implanted defibrillator may not be the ideal solution for patient care.

### CROSS REFERENCE TO RELATED APPLICATIONS

This application is part of scientific work that originated in conjunction with provisional patent filed Dec. 18, 2002, (patent office reference number 60/434,128).

### BACKGROUND OF THE INVENTION

1. Field of the Invention

The present invention relates generally to devices used to terminate arrhythmia using electrical cardioversion. The device is designed to facilitate the creation of various shock vector configurations between internal and external electrodes using a novel vector directing and patient protection junction box.

2. Description of the Prior Art

Recent statistics indicate that approximately ¾ million Americans will undergo Electrophyisology studies this year and that this number is expected to rise dramatically because of the aging population. The expected rise is increased further with the new scientific understanding of the impact that Atrial fibrillation and Atrial Flutter have on the cause or aggravation of other diseases such as stroke and of heart failure. As a result, additional measures are needed to help save lives but also to eliminate life debilitating injury, such as that caused by stroke, early stages of heart failure and reduced physical exertion capacity caused by Atrial fibrillation because 25% of the hearts blood pumping capacity is lost.

The art and science of electrical cardioversion is not new nor is the use of transvenouse leads or patches rather than paddles for delivering the electrical energy to cardiovert. Early teachings can be found in patents by Stoft et al. (U.S. Pat. No. 3,566,876 Mar., 1971), Jaros et al (U.S. Pat. No. 3,605,754 Sep. 29, 1971), Mirowski et al. (U.S. Pat. Nos. 3,614,955 Oct., 1971 and 3,942,536 Mar. 9, 1976), Heilman et al (U.S. Pat. No. 4,270,549 June, 1981) and others. Therefore the generic methodology and or apparatus art work can be reference when considering system that are designed to deliver electrical energy for cardioversion using paddles or patches or leads in combination with subcutaneous patches or wire meshes. However little work has been done using a clinically viable and relevant system that allows for easy coupling to existing in hospital defibrillators or a defibrillator that is designed and equipped to deliver low energies on demand separate from the rescue shock high energy external defibrillation. Levy used a catheter to a metal plate shock vector for terminating Chronic Atrial Fibrillation in 1998 and published his work; reference High energy transcatheter cardioversion of Chronic Atrial Fibrillation; Levy S, Lauribe P, Dolla; Circulation 1988; 12:514-8. Additional work published by Levy also compared internal cardioversion against external in a randomized trail that showed reduced energy levels with increased cardioversion success for internal cardioversion the published work titled “A Randomized Comparison of Internal and External Cardioversion of Chronic Atrial Fibrillation” Levy S, Lacombe P, Cointe R, Bru P.; Journal of the American College of Cardiology 1992; 86;1415-20. The teachings defined the methodology of placing catheters in the right atrium and coronary sinus to form a vector for cardioversion suitable for cardioversion of Atrial Fibrillation. The work also published be Alt and others continued to expand on the teaching of Levy with respect to using standard electrophysiology like catheters for the purpose of internal cardioversion so that a step in the science of cardioversion electrodes was skipped or otherwise missed. Specifically the art and science of the cardioversion electrodes and catheter properties required to make the device clinically usefull, safe and effective. The connection of diagnostic catheters to field defibrillator designed to deliver as much as 400 Joules is possible with a basic cable. However if the user accidentally uses an energy that is not proper for the device and with proper calculations for current densities, system impedance, and energy dispersion the result can be burning and ablation.

### SUMMARY OF THE INVENTION

It is an object of the present invention is to provide a device and method which will terminate arrhythmia of the heart using electrical energy coupled between at least two electrodes, ideally designed to carry cardioversion energy and located within the body or located inside and outside of the body. A junction box that is selectable to varied configurations including a normal external shock configuration for rescue shock back-up in the event a catheter is inadvertently moved and therefore vector(s) required to terminate arrhythmia may have been compromised.

Another object of the present invention is to teach the varied vectors that can be achieved using the junction box system and catheter with multiple electrodes or several catheters and or single patch electrodes.

Another object of the invention is to discuss the novel design features of the patient protective circuitry built into the junction box that limits the amount of energy the patient can receive to the internal electrodes when an accidental high energy shock is coupled from the defibrillator.

Additional objects, features and advantages will be apparent in the written description which follows.

### BRIEF DESCRIPTION OF THE DRAWINGS

FIG. 1 is one possible embodiment of the design that has threeselections that have two distinct high voltage shock vector configurations. The first being a shock between two internal electrodes that are in-direct contact with the myocardium. The second setting being configured so that a bi-directional shock vector is configured by directing current flow to one of the internal electrodes and also to one of the external (located outside the body) electrodes. The third being only an external shock configuration whereby the two external electrodes allow current flow between them so as to allow standard “life saving” defibrillation or cardioversion therapy to be applied. The first and second selection having protective circuitry that eliminates the possibility of energy greater than (1200 VDC) to be delivered to patient when any internal electrode is active. The third selection having no affect at all on the amount of energy delivered and only acts as a “selectable” passive conduit to the patient so that very high energy (>1200 VDC) shocks can be delivered between two external electrodes. The two external electrodes preferably positioned in the posterior and anterior position as shown in FIG. 13 with Anterior electrode preferable being the active external electrodes when using in combination with internal electrode(s). However, both external electrodes can be made to be active and is obvious disclosure to those versed in the art.

The system would include any cables and or test devices required to effectuate the connection, pre-testing and or clinical utility as is know in the art or specific to the system here in disclosed.

FIG. 2 shows an internal only shock configuration, through switch box (protected circuit), whereby the shock vector is between the catheter in the Right Ventricle and the catheter in the Right Atrium.

FIG. 3 shows an internal only shock configuration, through switch box (protected circuit), whereby the shock vector is between the catheter in the Right Atrium and the catheter in the Coronary sinus.

FIG. 4 shows an internal only shock configuration, through switch box (protected circuit), whereby the shock vector is between the catheter in the Pulmonary Artery and the catheter in the Right Atrium.

FIG. 5 shows an internal only shock configuration, through switch box (protected circuit), whereby the shock vector is between three electrodes on a single catheter having a electrodes located in the Pulmonary Artery, Right Atrium and Right Ventricle and whereby the resulting shock vector is bidirectional. Specifically one electrode provides the source of current and the other two electrodes are return paths or two electrodes provide a source of current and one electrode becomes a return path.

FIG. 6 shows a shock configuration, through switch box (protected circuit), whereby the shock vector is between the catheter in the Right Ventricle and the catheter in the Right Atrium and the paddle (gel pad) located on the patients chest. The shock is therefore bi-directional and vectors between one internal electrode to the other internal electrode plus the external electrode provide one source of current and two return paths in two very different planes that form vector paths of 15 to 90 degrees difference between vectors. The increased separation of the shock vectors improves the amount of myocardium captured and stimulated causes it to function normally (Sinus Rhythm due to cardioversion or defibrillation).

FIG. 7 shows a shock configuration, through switch box (protected circuit), whereby the shock vector is between the catheter in the Coronary sinus and the Right Atrium and also the paddle (gel pad) located on the patients chest. The shock is therefore bi-directional and vectors between one internal electrode to the other internal electrode plus the external electrode provide one source of current and two return paths in two very different planes that form vector paths of 15 to 90 degrees difference between vectors. The increased separation of the shock vectors improves the amount of myocardium captured and stimulated causes it to function normally (Sinus Rhythm due to cardioversion or defibrillation). The use of Coronary sinus with for one vector provides for more of the Atria (both atrium left and right) to be captured when using this configuration.

FIG. 8 shows a shock configuration, through switch box (protected circuit), whereby the shock vector is between the catheter in the Pulmonary Artery and the Right Atrium and also the paddle (gel pad) located on the patients chest. The shock is therefore bi-directional and vectors between one internal electrode to the other internal electrode plus the external electrode provide one source of current and two return paths in two very different planes that form vector paths of 15 to 90 degrees difference between vectors. The increased separation of the shock vectors improves the amount of myocardium captured and stimulated causes it to function normally (Sinus Rhythm due to cardioversion or defibrillation). The use of Coronary sinus with for one vector provides for more of the Atria (both atrium left and right) to be captured when using this configuration. This selection may be used when placement into Coronary Sinus is difficult or in the absence of x-ray flouroscopy equipment.

FIG. 9 shows an internal only shock configuration, through switch box (protected circuit), whereby the shock vector is between three electrodes on a single catheter having a INTERNAL electrodes located in the Pulmonary Artery, Right Atrium and Right Ventricle and the External electrode is located on the patients chest. Whereby the resulting shock vector is tri-directional and in deed 3 dimensional. Specifically one electrode(s) provide(s) the source of current and the other two electrodes are return paths or three electrodes, preferably all three internal, provide a source of current and one electrode, external, becomes a return path. The vector formed is three dimensional and generally conical and capable of capturing and stimulating a large mass of the heart myocardium and would include a large portion of both Atria and Right ventricle. The use of only two internal electrodes in this configuration coupled to the external electrode creates a bi-directional vector.

FIG. 10 shows an external only shock configuration, through switch box (passive unprotected circuit side) that allows the delivery of high energy through paddles or gel pads.

FIG. 11 shows a shock configuration, through switch box (protected circuit), whereby the shock vectors are between the catheters in the Right Atrium and the Right Ventricle and propagate thru the heart to the paddle (gel pad) located on the patients chest. The shock therefore has two distinct vector angles formed between two internal electrodes and one or more external electrodes. The configuration is ideally suited to capture large Ventricular and Atrial mass for terminating VT, VF and SVT's during EP studies and ablation procedures.

FIG. 12 shows a shock configuration, through switch box (protected circuit), whereby the shock vectors are between the catheters in the Right Atrium and propagate thru the heart to the paddle (gel pad) located on the patients chest. The shock therefore has one distinct vector angles formed between the internal electrode and one or more external electrodes. The configuration is ideally suited to capture large Atrial mass for terminating AF udring during EP studies and ablation procedures and for easily and safely terminating Chronic Refractory Atrial Fibrillation (AF that has failed cardioversion using drugs and external cardioversion).

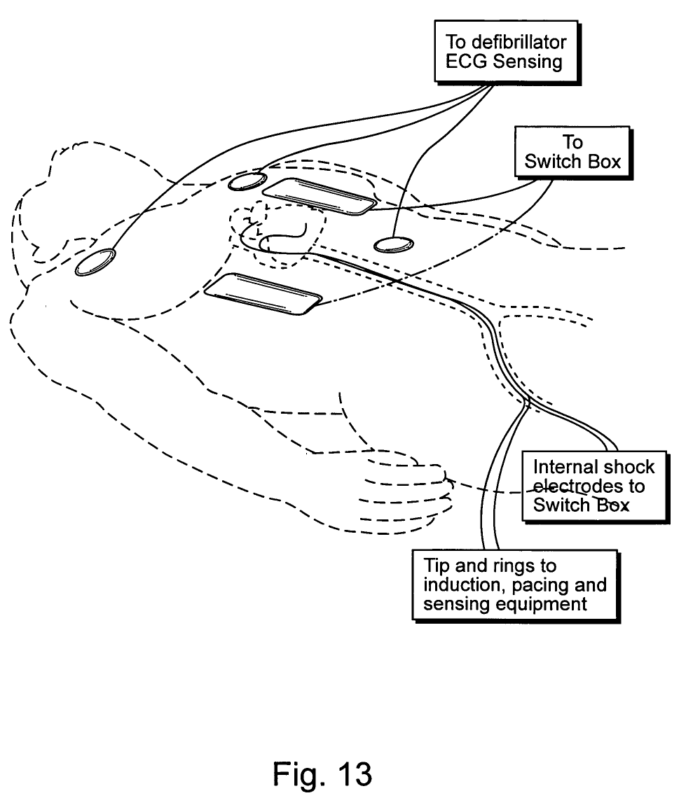

The technique or method taught in FIG. 12 could be used with only one electrode in the Right atrium (without having a catheter in Right ventricle) couples to a single external electrode only that would form an energy vector through both Atria. The ECG electrodes on the chest of patient would be used to synchronize shock with QRS complex. Additionally, the use of low energy (<50 J) means that anesthesia and intubation is not required. Deep sedation or and conscious sedation can be utilized which reduces the need for specialized staff to conduct what should be a routine procedure.

### DETAILED DESCRIPTION OF THE INVENTION

APPARATUS

The vector selectable junction box with patient protective circuit (safety) that couples to standard field external defibrillators (Safe Junction Box) will be used in discussions. The Safe Junction box provides for a circuit that is activated once an unsafe energy is reached and redirects the excess energy away from the patient.

The catheter mounted cardioversion electrode is another term to be used in discussions and ideally describes an electrode that has an ideal cardioversion electrode surface area because less than 2 Amps per centimeter squared is achieved by the design of electrode and protective circuit within switch box.

The catheter mounted cardioversion electrode is another term to be used in discussions and ideally describes an electrode that has an ideal cardioversion electrode surface area because less than 2 Amp per centimeter squared is achieved by design of electrode and protective circuit within switch box.

The external (cutaneous) “patch” electrode is another term to be used in discussions and ideally describes an electrode as commonly known in the art of external defibrillation. The patch being ideally designed for high-energy external cardioversion (>50 Joules) but also capable of coupling to low energy.

The Safe Junction Box, in one preferred form, comprises a high performance and high durability switch designed to switch energy as low as 0.1 milli Volts up to 10,000 volts and selectable to 3 optional circuit paths. The switch must be designed to comply with all UL and IEC-601 requirements but must be ideally designed not increase resistance and or impedance to defibrillation voltage coupled from external defibrillator.

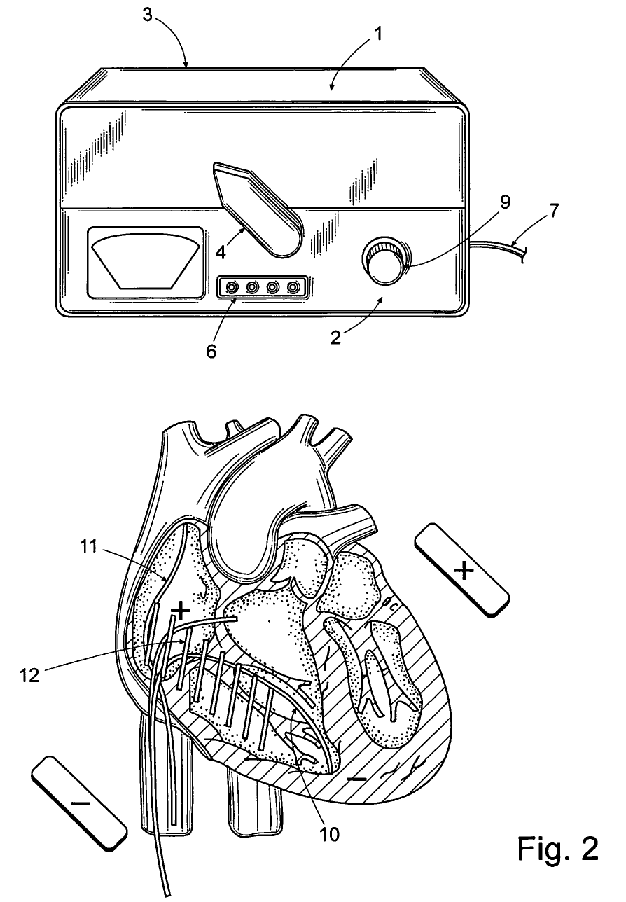

The apparatus of the invention will first be described with reference to FIGS. 1 and 2, one preferred embodiment, is a front view of the Safe Junction Box. The switch box consisting of electrically isolated housing 1 with electrically isolated face plate 2 and electrically isolated back plate 3 (not seen but directly behind 2) that forms the isolated casing. The Safe Junction Box includes a high voltage switch 4 selectable to three positions that directs energy from the defibrillator through the patient protective circuitry or away from the patient protective circuitry when external (back-up) defibrillation is desired. The internal workings of the switch box being designed with semi-conductor technology (not shown) applied to high voltage application that “passively” senses voltages so that they do not exceed 1000 VDC and if they do it diverts the balance of voltage to a secondary path that absorbs the voltage. The circuit is not designed to stop unsafe voltage but designed to limit the amount of voltage so patient always gets energy required for cardioversion but circuit controls maximum amount of energy so that electrode heating does occurs at the surface, otherwise known and as ablation. So the Safe Junction Box can change the desired clinical result by either controlling voltage for safe cardioversion when electrode is located within the heart and also for high voltage (unprotected side of circuit) for external cardioversion. Additionally, a defibrillator input connector 6 mounted on the switch box so that any external defibrillator can be connected by using an adaptor cable (not shown) for each of the brands available in the field in any specific region of the world.

The energy coupled into Safe Junction Box through connector 6 and directed by switch 4 to either a protected circuit or unprotected circuit that allows energy from defibrillator to pass unmodified. The energy from either protected circuit or unprotected circuit directed to connectors found on the back 3 or front plate 2 of switch box for catheter electrode connections or cutaneouse patch or paddle (external electrode) connection 9.

In FIG. 2 one preferred embodiments is shown in the “as used” setting in the heart. A catheter having a high surface electrode 10 is located in right ventricle and a second catheter having a high surface electrode 11 is located right atrium. The energy provided by external defibrillator forming a single dimension vector 12 that is ideally suited to terminated ventricular fibrillation or ventricular tachycardia.

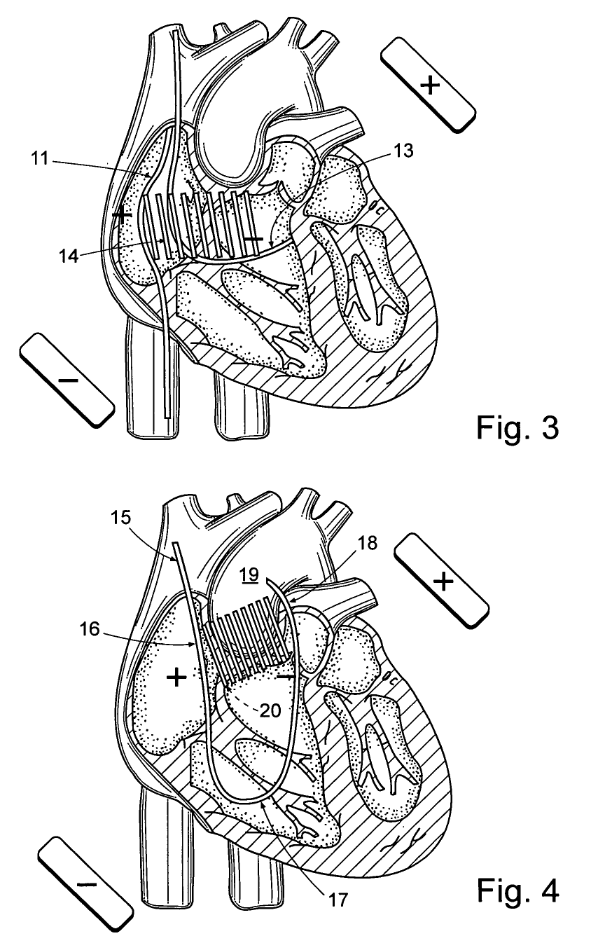

In FIG. 3 another preferred embodiments is shown in the “as used” setting in the heart. A catheter having a high surface electrode 11 is located right atrium and a second high surface electrode 13 is located in the coronary sinus. The energy provided by external defibrillator forming a single dimension vector 14 that is ideally suited to terminate atrial fibrillation atrial flutter and atrial tachycardia.

In FIG. 4 another preferred embodiments is shown in the “as used” setting in the heart. A catheter having 3 high surface electrodes 15 is positioned so one electrode is located in right atrium 16 a second high surface electrode right ventricle 17 and a third is located in the pulmonary artery 18. The energy provided by external defibrillator forming a single dimension vector between 16 and 18 resulting in a single dimension vector 19 that is ideally suited to terminate atrial fibrillation atrial flutter and atrial tachycardia. Alternatively in a single dimension vector formed between 16 and 17 that is ideally suited to terminated ventricular fibrillation or ventricular tachycardia. All done by using a single catheter that triangulates the heart in a quasi-Ivanhoe triangle configuration.

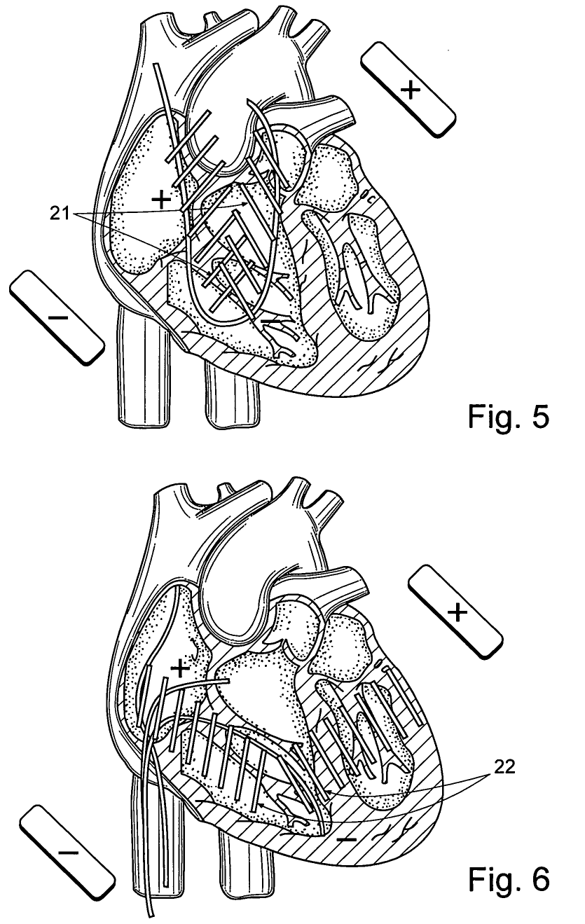

FIG. 5 is the same as FIG. 4 except that a two-dimensional shock 21 that is ideal for terminating ventricular fibrillation or ventricular tachycardia is created.

FIG. 6 is the same as FIG. 2 except that a two dimension shock 22 is created and ideal for terminating ventricular fibrillation or ventricular tachycardia

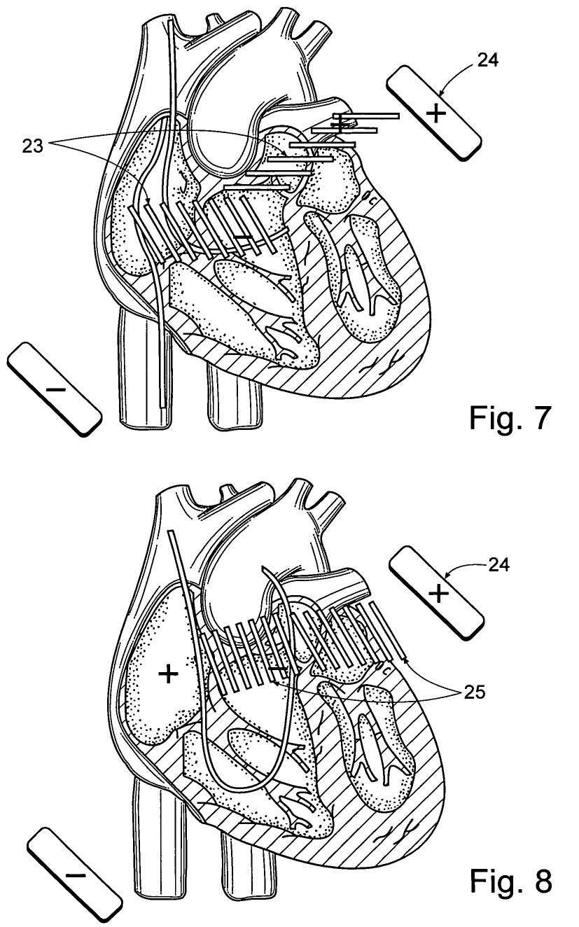

FIG. 7 is the same as FIG. 3 except that a two dimension shock 23 is created and ideal for terminating atrial fibrillation atrial flutter and atrial tachycardia because energy captures the left atria. The cutaneuose patch 24 is made active via the switch box yet the other patch remains inactive so that the energy vector is formulated by Safe Junction Box.

FIG. 8 is the same as FIG. 4 except that a two dimension shock 25 is created and ideal for terminating atrial fibrillation atrial flutter and atrial tachycardia because energy captures the left atria. The cutaneuose patch 24 is made active via the switch box yet the other patch remains inactive so that the energy vector is formulated by Safe Junction Box.

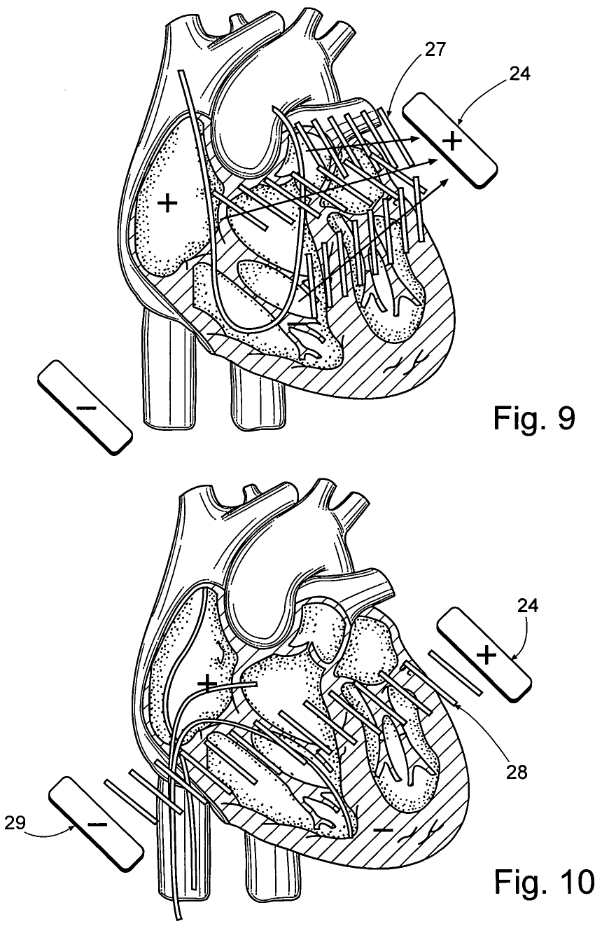

FIG. 9 is the same as FIG. 4 except that a three dimensional shock 25 is created and ideal for terminating all arrhythmia because energy captures all four chambers of the heart and primarily the left side of heart. The cutaneuose patch 24 is made active via the switch box yet the other patch remains inactive so that the three dimensional energy vector is formulated by Safe Junction Box. The catheter electrodes are all made active and of the same polarity with only a single cutaneouse patch being made the opposite polarity and therefore a three dimensional vector is formed.

FIG. 10 shows how the energy can be converted back to standard external defibrillation mode when the switch 4 is turned to proper location and a high energy vector is created when the cutaneuose patch 24 is made active with one polarity and the other cutaneuose patch 29, is made active with opposite polarity to form a “standard” external defibrillation shock vector 28.

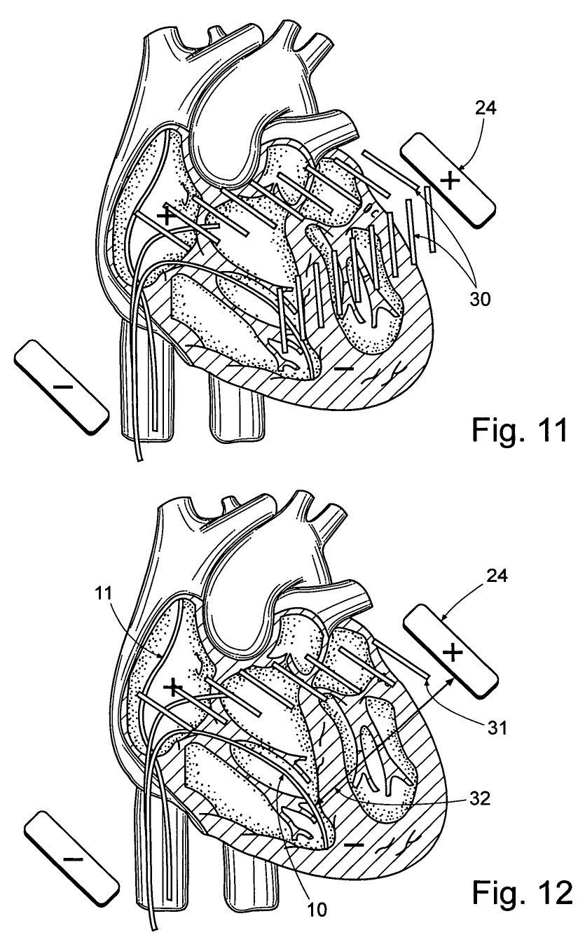

FIG. 11 is the same as FIG. 2 except that a two single dimension shocks with none uniform alignment 30 is created and ideal for terminating any arrhythmia of the heart.

FIG. 12 is the same as FIG. 2 except that a one single dimension shocks with none uniform alignment 31 is created and ideal for terminating atrial fibrillation atrial flutter and atrial tachycardia because energy captures the left atria. The shock vector travels through the entire heart muscle since it is trying to couple to the cutaneouse patch through the outer wall of left atria. Additionally, FIG. 12 teaches by obvious pictorial description that catheter in right atrium can be made inactive and that catheter in left ventricle can be made electrically active so that a single dimensional vector 32 can be created and is ideal for terminating ventricular fibrillation or ventricular tachycardia.

FIG. 13 shows one possible “as used” configuration of the system that utilizes EKG pads directly from the defibrillator to synchronize the shock for any tachycardia, fibrillation or flutter.

A cutaneouse electrode (patch, paddle or similar device) is located on the back of the patient 13 and a second cutaneouse electrode (patch, paddle or similar device) is located on the chest of the patient 14. The cutaneouse electrode located on the chest made active by the Safe Junction Box and also the high surface area electrodes located on the heart. The energy of defibrillator

1. A system for use with standard defibrillators that includes high surface area electrodes designed to be used internally or externally to form a variety of shock vector configurations better described as single dimension, two dimension and also three dimensional, the system consisting of:
i. a safe junction box equipped with passive semi-conductor like circuit designed to protect patient from unsafe voltages by diverting the excess and only the excess energy away from the electrodes but only in certain selection settings;
ii. a switch mounted on the safe junction box designed to redirect the energy supplied by the defibrillator to one or more electrodes depending on choice that will always include at least two and at least the option to switch back to external for safety; the second option being directed through patient protective circuitry that limits the voltage but never stops all the voltage to ensure therapy is delivered to terminate life threatening arrhythmia or none life threatening arrhythmia;
iii. internal electrodes mounted on a catheter or lead made so that current densities to be used at the surface of the electrodes never exceed 2 amp per centimeter squared;
iv. external electrodes mounted on the skin of the patient and requiring no blood or other internal body fluid contact; and
v. the entire system being passive in nature because all energy required to defibrillate is supplied by another box (standard field defibrillator) or integrated with its own pulse generating circuitry that has been designed to isolate internal shocks from external shocks so that a patient can never be accidentally be shocked with energies that would result in the electrodes located in the heart having greater than 2 amps per centimeter squared of energy.

2. The system of claim 1, wherein a catheter is located in the right atria and a separate catheter is located in the right ventricle and a shock vector is created for terminating ventricular tachycardia or ventricular fibrillation.

3. The system of claim 1, wherein a catheter is located in the right atria and a separate catheter is located in pulmonary artery and a shock vector is created for terminating atrial tachycardia or ventricular fibrillation.

4. The system of claim 1, wherein a single catheter is provided with three electrodes and said electrodes are located in the right atria, right ventricle and pulmonary artery and a shock vector is created for terminating atrial tachycardia or ventricular fibrillation, the direction of the shock vectors being varied using the safe switch box selector knob that redirects the path of energy.

5. The system of claim 1, wherein a single catheter with two electrodes and said electrodes is provided are located in the right atria and coronary sinus and a shock vector is created for terminating atrial tachycardia or atrial fibrillation.

6. The system of claim 1, where one of the preferred embodiments for managing unsafe energy is a metal oxide varistor that limits energy by way of limiting voltage for the internal electrodes, the desired limit being adjustable by properly specifying the metal oxide varistor so that surface area current densities never exceed 2 amps per centimeter squared.
